(19) United States
(12) Patent Application Publication
(10) Pub. No.: US 2025/0271429 A1
(43) Pub. Date: Aug. 28, 2025

(54) COMBINATION ASSAY OF CYTOKINES AND HUMAN ANTIBODIES TO MAP FOR THE DIAGNOSIS OF CROHNS DISEASE, TUBERCULOSIS, AND OTHER BACTERIAL DISEASES

(71) Applicants: John Todd Kuenstner, Weatherford, TX (US); Jean-Michel Galarneau, Calgary (CA)

(72) Inventors: John Todd Kuenstner, Weatherford, TX (US); Jean-Michel Galarneau, Calgary (CA)

(21) Appl. No.: 19/059,577

(22) Filed: Feb. 21, 2025

Related U.S. Application Data
(60) Provisional application No. 63/556,688, filed on Feb. 22, 2024.

(51) Int. Cl. G01N 33/569

(52) U.S. Cl. CPC G01N 33/5695; G01N 2333/35; G01N 2333/54; G01N 2333/5421; G01N 2333/57; G01N 2469/20

(57) ABSTRACT

A system or method for a combination assay of human antibodies to Mycobacterium avium subsp. paratuberculosis (MAP) and cytokines for the diagnosis of Crohn's disease, tuberculosis, and other bacterial diseases in symptomatic and asymptomatic individuals is provided herein. The system or method describes generally using a combination of human antibodies to MAP useful for the detection of a MAP infection in human blood samples and cytokines secreted by the human host with a MAP infection to provide a simple and rapid serological test which can diagnose patients with Crohn's disease and can aid in the selection of patients for certain antibiotic therapies. A similar system could be used for the diagnosis and selection for therapy of tuberculosis and other mycobacterial or bacterial diseases.

### CROSS-REFERENCE TO RELATED APPLICATIONS

This patent application claims the benefit of U.S. Provisional Patent Application Ser. No. 63/556,688, filed on Feb. 22, 2024, entitled “Combination MAP Antibody and Cytokine Assay for the Diagnosis of Crohn's Disease, Tuberculosis, and Other Bacterial Diseases.”

### BACKGROUND OF THE INVENTION

This patent application generally relates to a combination assay consisting of cytokines and human antibodies to MAP (or another mycobacterium) for the diagnosis of Crohn's disease, tuberculosis, and other bacterial diseases in symptomatic and asymptomatic individuals.

This patent application describes generally using a combination of human antibodies to MAP useful for the detection of a MAP infection in human blood samples and cytokines secreted by the human host with a MAP infection to provide a simple and rapid serological test which can diagnose patients with Crohn's disease, tuberculosis, and other bacterial diseases, and can aid in the selection of patients for certain antibiotic therapies.

In 1895, Johne and Frothingham first described a chronic diarrheal disease of cattle (now known as Johne's disease (JD)) (Johne H A, Frothingham L. Ein Eigenthuemlicher Fall von Tuberculose beim Rind. Deutsche Zeitschrift fuer Tiermedizin und Vergleichende Pathologie. 1895;21:438-54) and by 1912, Twort et al identified and cultured the bacterium, now known as Mycobacterium avium ssp. paratuberculosis (MAP), which causes this disease (Twort F W, Ingram G L, Ingram Y. A method for isolating and cultivating Mycobacterium enteridis chronicae pseudotuberculosae bovis johne and some experiments on the preparation of a diagnostic vaccine pseudotuberculosae enteritis of bovines. Proc Royal Soc Lond. 1912; 84:517-43). Kennedy Dalziel First speculated in 1913 that MAP also causes Crohn's disease (CD) (Dalziel T K. Chronic Interstitial Enteritis. Br Med J. 1913;2:1068-70), which he called “chronic interstitial enteritis”. JD is a chronic diarrheal disease of cattle, a wasting disease in sheep and a pathogen in most wildlife species (Over K, Crandall PG, O'Bryan C A, et al. Current perspectives on Mycobacterium avium subsp. paratuberculosis, Johne's disease, and Crohn's disease: A review. Crit Rev Microbiol. 2011; 37:141-156).

The culture of MAP from the tissue of patients with CD is notoriously difficult and many investigators have failed to demonstrate the organism, but meta-analyses have shown that most patients with CD have MAP in their gastrointestinal tissues (Feller M, Huwiler K, Stephan R, Altpeter E, Shang A, Furrer H, Pfyffer G E, Jemmi T, Baumgartner A, Egger M. Mycobacterium avium subspecies paratuberculosis and Crohn's disease: a systematic review and meta-analysis. Lancet Infect Dis 2007; 7:607-613 [PMID: 17714674] and Abubakar I, Myhill D, Aliyu S H, Hunter P R. Detection of Mycobacterium avium subspecies paratuberculosis from patients with Crohn's disease using nucleic acid-based techniques: a systematic review and meta-analysis. Inflamm Bowel Dis 2008; 14:401-410 [PMID: 17886288]).

Based on the foregoing evidence and similarities of the signs and symptoms of CD and JD, an ever-increasing number of medical scientists (Kuenstner J T, Naser S, Chamberlin W, Borody T J, Graham D Y, McNees A, Hermon-Taylor J, Hermon-Taylor A, Dow C T, Thayer W, Biesecker J, Collins M, Sechi L, Singh S V, Zhang P, Shafran I, et al. The Consensus on Mycobacterium avium ssp. paratuberculosis (MAP) 2017. Frontiers in Public Health September 2017; 5: article 208. doi: 10.3389/fpubh.2017.00208) have concluded that MAP is the cause of most cases of CD. The RedHill Biopharma FDA phase II/III clinical trial of combination antimycobacterial therapy for CD supports the thesis that MAP causes CD (Graham D, Hardi R, Welton T, et al. Phase III randomized, double-blind, placebo-controlled, multicenter, parallel-group study to assess the efficacy and safety of add-on fixed-dose anti-mycobacterial therapy (RHB-104) in moderately to severely active Crohn's disease. United Eur Gastroenterol. 2018; 6:1586) and (Graham David Y., Naser Saleh A., Borody Thomas, Hebzda Zbigniew, Sarles Harry, Levenson Scott, Hardi Robert, Arlukowicz Tomasz, Svorcan petar, Fathi Reza, Bibliowicz Aida, Anderson Patricia, McLean Patrick, Fehrmann Clara, Harris M. Scott, Zhao Shuhong, Kalfus ira N. Randomized, Double-Blind, Placebo-Controlled Study of Anti-mycobacterial Therapy (RHB-104) in Active Crohn's Disease. Antibiotics 2024, 13, 694.)

Recently, a multi-laboratory, prospective controlled study employing blind testing of whole blood samples by multiple culture techniques, found widespread viable MAP bacteremia in the 61 CD patients and 140 non-CD control subjects. Approximately 78% of CD patients had blood cultures positive for MAP. Nine subjects with a positive MAP Phage assay (4/9) or MAP culture (5/9) were again positive with the MAP Phage assay one year later (Kuenstner J T, Potula R, Bull T, et al. Presence of infection by Mycobacterium avium subsp. paratuberculosis in the blood of patients with Crohn's disease and control subjects shown by multiple laboratory culture and antibody methods. Microorganisms, 2020; 8:2054).

In the foregoing study, of the serological methods, the best antibody for identifying CD patients from non-CD controls was the Hsp65 antibody (Peilin Zhang, U.S. Pat. No. 10,656,153 entitled, “Diagnostic Method and Devices for Autoimmune Disease,” granted Feb. 23, 2018). In the Zhang patent, he showed that, in addition to the MAP organism, other bacteria could be cultured from the blood of CD patients. The blood of these CD patients had antibodies associated with these other bacteria including, RPOB, EF-G and EF-Tu.

Furthermore, the 78% of CD patients with MAP positive blood cultures showed cytokine expression patterns similar but not identical to cytokine expression patterns of patients with tuberculosis infection. (Kuenstner J T, Xu Q, Bull T, Foddai A C G, Grant I R, Naser S, Potula R, Zhang P, Shafran I, Akhanli S A, Khaiboullina S, Kruzelock R. Cytokine expression in subjects with Mycobacterium avium ssp. paratuberculosis positive blood cultures and a meta-analysis of cytokine expression in Crohn's disease. Frontiers in Cellular and Infection Microbiology, 14:1327969.2024 doi: 10.3389/fcimb.2024.1327969).

What is needed is a system or method that allows for the rapid diagnosis of individuals with CD, tuberculosis, or other bacterial diseases, that improves upon the prior art or provides other advantageous features. Other features and advantages will be made apparent from the present specification. The teachings disclosed herein extend to those embodiments that fall within the scope of this specification and the claims, regardless of whether they accomplish one or more of the needs described herein.

### BRIEF DESCRIPTION OF THE FIGURES

This application will become more fully understood from the following summary and description, taken in conjunction with the accompanying figures, wherein like reference numerals refer to like elements, in which:

FIG. 1 is a table describing the sample of antibodies measured in the 199 participants stratified by disease status (CD vs. non-CD).

FIG. 2 is a table describing the classification statistics of each antibody in both continuous and binary forms following the published cut-off values and those derived from the data.

FIG. 3 is a table describing the results of multivariable logistic regression with the highest AUC combining the best two antibodies.

FIG. 4 is a graph representing the area under the receiver operating curve (AUC) derived from the final, multivariable logistic regression as shown in the bottom panel of FIG. 3.

FIG. 5 is a flowchart representing at least one embodiment of the presently claimed invention.

FIG. 6 is an aerial view of an embodiment of the presently claimed invention as a cytokine assay kit.

FIG. 7 is a close-up transparent view of one well of the cytokine assay kit of FIG. 6.

FIG. 8 is a sample depiction of a panel array result from use of the cytokine assay kit of FIGS. 6 and 7.

### SUMMARY OF THE INVENTION

It is believed that MAP infection is the cause of most cases of CD and that therapy which effectively targets MAP in the infected human host will result in cure or profound remission. To identify MAP infection and CD in patients with the classic symptoms and signs of CD, we used antibodies corresponding to antigens (i.e. PknG, PtpA and Hsp65) produced by the MAP organism combined with cytokines (i.e. IFNg, IL-8 and IL-17a) secreted by the human host. This combination may be used for a simple and rapid serological test which will select CD patients who can be treated with anti-MAP antibiotics such as those used in the RedHill Biopharma FDA clinical trials for CD patients. (Graham David Y., Naser Saleh A., Borody Thomas, Hebzda Zbigniew, Sarles Harry, Levenson Scott, Hardi Robert, Arlukowicz Tomasz, Svorcan Petar, Fathi Reza, Bibliowicz Aida, Anderson Patricia, McLean Patrick, Fehrmann Clara, Harris M. Scott, Zhao Shuhong, Kalfus ira N. Randomized, Double-Blind, Placebo-Controlled Study of Anti-mycobacterial Therapy (RHB-104) in Active Crohn's Disease. Antibiotics 2024, 13, 694).

This patent application is based on the results of a prospective, case-control laboratory test study of 199 subjects (61 CD patients and 138 non-CD controls). Human antibodies to MAP, including Hsp65, PknG, PtpA, CL1, and MAP IDEXX, were measured under blind conditions in the plasma of the 199 subjects (Kuenstner J T, Xu Q, Bull T, Foddai A C G, Grant I R, Naser S, Potula R, Zhang P, Shafran I, Akhanli S A, Khaiboullina S, Kruzelock R. Cytokine expression in subjects with Mycobacterium avium ssp. paratuberculosis positive blood cultures and a meta-analysis of cytokine expression in Crohn's disease. Frontiers in Cellular and Infection Microbiology, 2024).

The study protocol was reviewed on 20 Oct. 2017 by the Temple University IRB (IRB protocol #24790). Further details on the study design and participants, diagnosis and diagnostic categorization, and the procedures have been described previously (Kunstner J T, Potula R, Bull T, Grant I, Foddai A, Naser S, Bach H, Zhang P, Yu D, Lu X, and Shafran I. Presence Of Infection By Mycobacterium avium subsp. paratuberculosis In The Blood Of Patients With Crohn's Disease And Control Subjects Shown By Multiple Laboratory Culture And Antibody Methods. Microorganisms 2020, 8, 2054; doi: 10.3390/microorganisms8122054 and Kuenstner J T, Xu Q, Bull T, Foddai A C G, Grant I R, Naser S, Potula R, Zhang P, Shafran I, Akhanli S A, Khaiboullina S, Kruzelock R. Cytokine expression in subjects with Mycobacterium avium ssp. paratuberculosis positive blood cultures and a meta-analysis of cytokine expression in Crohn's disease. Frontiers in Cellular and Infection Microbiology, 2024).

To examine the association between each antibody and its classification ability in terms of correctly predicting CD cases, means and medians were first compared by cohort with both a t-test and a non-parametric Wilcoxon rank-sum test, making no assumptions about the data. Second, a bivariate analysis using logistic regression was undertaken to assess the association between the antibodies and the outcome and to compute classification statistics. To achieve this, logistic regression was fit to predict the outcome of CD in a complete-case analysis, using the continuous form of the variable representing the antibody and its binary analog using prescribed cut-offs either derived from these data (through the methods described herein) or from previously published studies. The intersection of the highest sensitivity and specificity relative to the predicted probability of being a case generated by the logistic regression was then identified to find the probability cut-off at which classification statistics could be computed. SEN, SPE, PPV, NPV, and the proportion of observations correctly identified (CC) along with the AUC were generated from the logistic regression. Odds ratios and p-values were also generated and presented.

Subsequently, a multivariable model was constructed from the best combination of two antibodies to test whether classification statistics could be improved. This process was iterative and multiple logistics regressions were fit. The same approach was undertaken in the bivariate analysis to compute classification statistics derived from the multivariable logistic regression.

Finally, to further improve on the model, the combination of the two most predictive antibodies was entered into a backward stepwise regression (p-value for entry=0.05, p-value for removal=0.1), locking these terms in the model and introducing a panel of 14 cytokines (Appendix A). A final multivariable was derived to compute classification statistics using the same approach discussed above, ignoring potential variance inflation issues and shrinkage. These were then compared to the classification statistics of the multivariable model of the two best antibodies. All work was performed in Stata (StataCorp. 2023. Stata Statistical Software: Release 18. College Station, TX: StataCorp LLC).

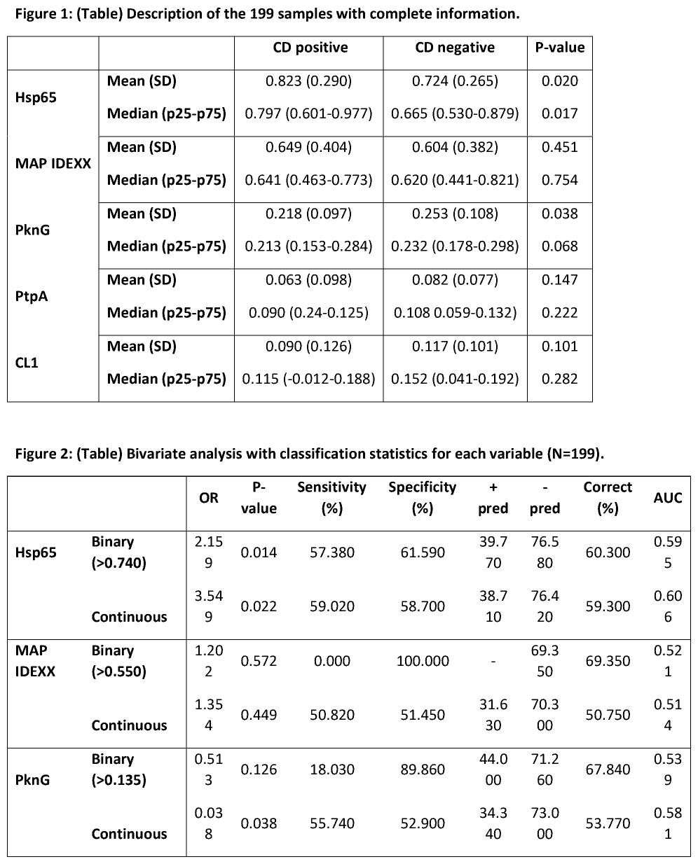

Of the 201 participants eligible for this analysis, 199 had data on all five antibodies and were retained for final analysis, resulting in 61 CD cases and 138 controls. FIG. 1 describes the sample of antibodies measured in the 199 participants stratified by disease status (CD vs. non-CD).

Of the five antibodies tested, only Hsp65 and PknG showed differences between CD patients and non-CD controls, with Hsp65 showing the greatest differences where CD patients had significantly higher levels of the antibody (p=0.020) following a t-test and a Wilcoxon non-parametric rank-sum test. For PknG, this relationship was reversed where CD patients had significantly lower levels of this antibody (p<0.038) following a t-test and borderline significantly lower following a non-parametric rank-sum test. CD patients also had slightly higher levels of MAP IDEXX, but this difference was not significantly greater than zero, while both PtpA and CL1 were lower in CD patients than in non-CD controls, a mean difference not significantly greater than zero.

FIG. 2 shows the classification statistics of each antibody in both continuous and binary forms following the published cut-off values discussed earlier and those derived from the data where applicable. The highest AUC was calculated for Hsp65 (AUC continuous=0.606) and PknG (AUC continuous=0.581), corresponding largely with the results shown in FIG. 1. Both showed significant associations between their continuous form and CD with Hsp65 producing, for each unit increase in the antibody levels, a 255% increase in the odds of diagnosis (p=0.022), while this relationship was reversed for PknG where a 1 unit increase in antibody levels resulted in a 96% reduction in the odds of diagnosis (p=0.038). For MAP IDEXX, PtpA, and CL1, the binary form of the antibodies did not perform well, with AUCs slightly improving for CL1 and PtpA with their continuous analogs, albeit never producing significant results in logistic regressions. The highest sensitivity was again computed for Hsp65, while the lowest sensitivity was found in both the categorical forms of IDEXX and CL1, resulting in specificities of 100% at those cut-off values.

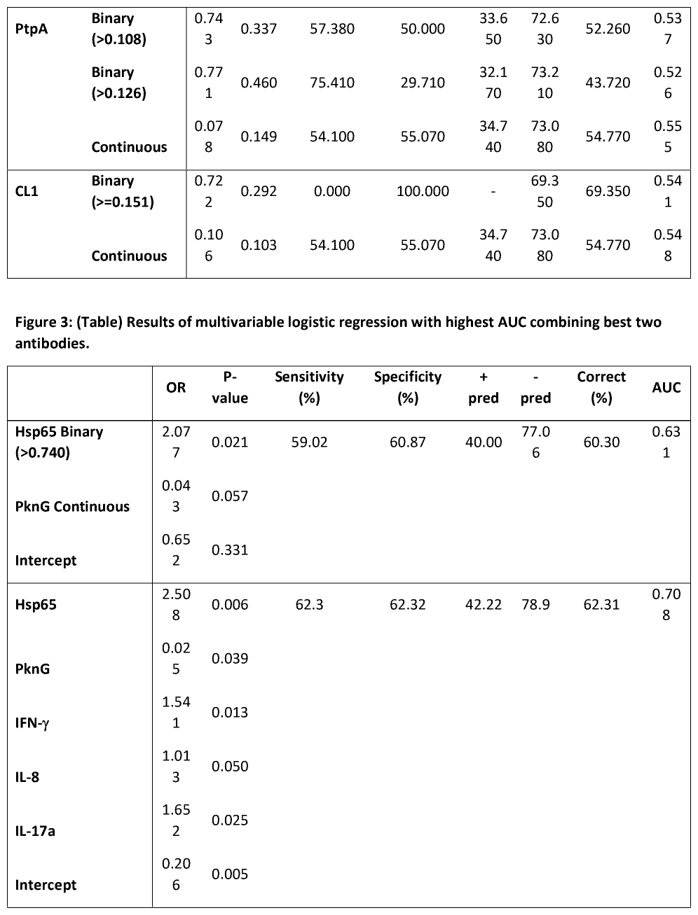

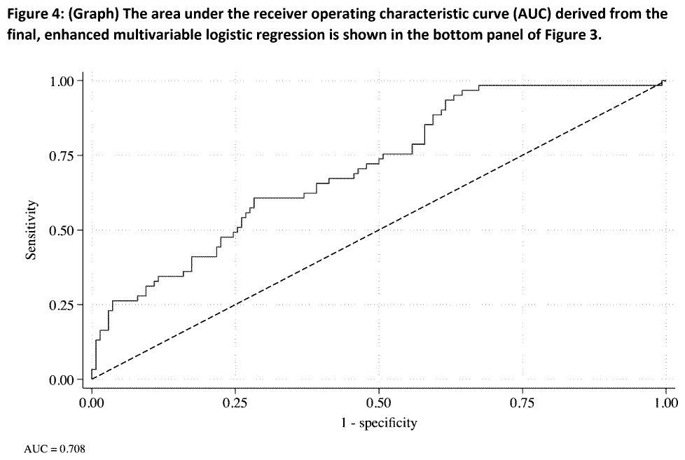

The best combination of two antibodies based on the highest AUC produced by a multivariable logistic regression is shown in the top panel of FIG. 3. The odds ratios for PknG and Hsp65 remain very similar to those shown in the bivariate table, with their statistical significance (p=0.021, p=0.057, respectively) remaining similar where PknG became borderline significant related to diagnosis. The sensitivity of the logistic regression was 59.02% with a specificity of 60.87% and an AUC of 0.631, approximately 1% and 5%, higher than the AUC produced by the antibodies independently, respectively (FIG. 1). The enhanced model, including the two best cytokines from a stepwise regression forcing in the two best antibodies, is shown in the bottom panel of FIG. 3. The odds ratios for both Hsp65 and PknG are similar to what is shown in the top panel, with their levels of significance increasing (p=0.006, p=0.039, respectively). The sensitivity of the model increased to 62.30%, and the specificity of the model decreased to 62.32%, while the AUC increased by more than 0.07 to 0.708 (FIG. 4). All three cytokines showed increases in the odds of diagnosis with an increase in the cytokine level with IFN-g and IL-17a significant at p<0.05, while IL-8 had a p-value of 0.050.

In conjunction with the direct culture and indirect culture methods (i.e. MAP phage assay), the human antibodies to MAP reported in this study bolster the evidence of infection in CD patients and may be used to select CD patients for anti-mycobacterial therapy.

This statistical analysis of five human antibodies to MAP determined in the 201 TU study subjects shows that a combination of human antibodies to MAP improves the SEN, SPE, PPV, NPV, and CC for CD diagnosis in comparison to any single human antibody to MAP. Further improvement in all these values was achieved using a combination of the human antibodies to MAP and three cytokines, typically elevated in CD, i.e., IL-8, IL-17a, and IFN-g.

If these MAP antibody tests are applied to a large sample with robust, healthy, and age-matched controls, the starting point is using a single human antibody to MAP, the Hsp65 antibody, which showed a SEN and SPE of 90.4% and 74.8%, respectively, in a sample using healthy Red Cross donors as controls. When tested in large samples with robust controls, incremental improvements of SEN, SPE, PPV, NPV, and CC value using combinations of the human antibodies to MAP should yield excellent CD prediction and/or corroboration of the disease suspected by the clinician.

It is anticipated that various combinations of human antibodies to MAP could be combined with other markers of CD activity, including C-reactive protein (CRP) and erythrocyte sedimentation rate (ESR), will also yield similar diagnostic test capability to the combination of human antibodies to MAP and cytokines which we have described in this patent application. Other yet unidentified cytokines may be elevated in CD and could be combined with human antibodies to MAP for improved CD diagnosis.

It is also anticipated that MAP antibody testing will be improved as new antibodies are discovered and that it will be possible to diagnose and treat patients for CD with an approach that combines a clinical history of typical signs and symptoms with these laboratory tests. These tests should ameliorate the difficulty in some cases of initially establishing the diagnosis of CD and reduce the use of invasive procedures required for CD diagnosis, such as endoscopy and biopsy.

The discovery relating to human infection by MAP could be adapted by persons of ordinary skill in the art to design similar tests for the diagnosis of tuberculosis, latent tuberculosis and mycobacterium tuberculosis (Mtb) non-infected status. Specifically, combinations of antibodies to tuberculosis such as Ag85B, Rv1860 and PstS1 and/or combinations of antibodies to Mycobacterium tuberculosis and cytokines elevated in TB (e.g. IFNg, IL-2, IL-5, IL-10, IL-17, GM-CSF and TNFa) could be used for the rapid diagnosis of active and latent tuberculosis (Dewii DNSS et al. Antibodies against native proteins of Mycobacterium tuberculosis can detect pulmonary tuberculosis patients. Scientific Reports. 13, article number 12685 (2023)).

Significantly improved test parameters are anticipated in future studies, which will include healthy and age-matched controls and solve for population issues including subjects in the control arm suffering from autoimmune diseases, including many that have been epidemiologically linked to MAP.

### DETAILED DESCRIPTION OF THE EXEMPLARY EMBODIMENTS

It should be understood that this application is not limited to the details or methodology set forth in the following descriptions or illustrated in the figures. It should also be understood that the phraseology and terminology employed herein is for the purpose of description only and should not be regarded as limiting.

In one embodiment, the diagnostic assay uses a combination of human antibodies to MAP such as Hsp65 and PknG.

In another embodiment, the diagnostic assay uses a combination of human antibodies to MAP such as Hsp65 and PknG, and at least three (3) cytokines, such as IL-8, IL-17a, and IFN-g.

In another embodiment, the diagnostic assay is a combination serological test based on human antibodies to MAP for the diagnosis and selection of CD patients for anti-MAP therapy.

In another embodiment, the diagnostic assay is a combination serological test based on Human antibodies to MAP and other bacterial antibodies, such as RPOB, EF-G, Hsp65, ATP5a, and EF-Tu, for the diagnosis and selection of CD patients for bacterial specific therapy.

In another embodiment, the diagnostic assay uses a combination of human antibodies to Mtb, such as Ag85B, Rv1860, and PStS1, and at least one cytokine typically elevated in the presence of tuberculosis, such as IFNg, IL-2, IL-5, IL-10, IL-17, GM-CSF, or TNFa, for the diagnosis of active or latent tuberculosis.

In another embodiment, the diagnostic test uses a combination of human antibodies to Mtb, such as Ag85B, Rv1860, and PStS1, for the diagnosis and selection of tuberculosis patients for anti-tuberculosis therapy.

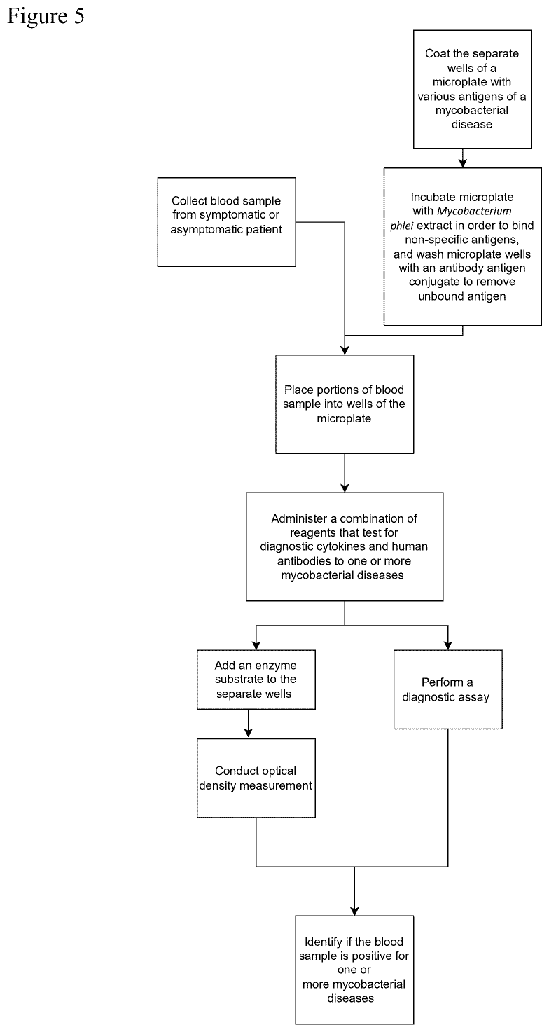

As shown in FIG. 5, in at least one embodiment of the invention, separate wells of a microplate are coated with various antigens of a mycobacterial disease; the microplate is then incubated with Mycobacterium phlei extract in order to bind non-specific antigens and wash the microplate wells with an antibody antigen conjugate to remove unbound antigen, while prior to or simultaneously a blood sample is collected from a symptomatic or asymptomatic patient. Portions of the blood sample are then placed into the wells of the microplate, after which a combination of reagents are administered that test for diagnostic cytokines and human antibodies to one or more mycobacterial diseases. While a diagnostic assay is performed, an enzyme substrate is added to the separate wells. An optical density measurement is then conducted. Finally, it is identified whether the sample is positive for one or more mycobacterial diseases.

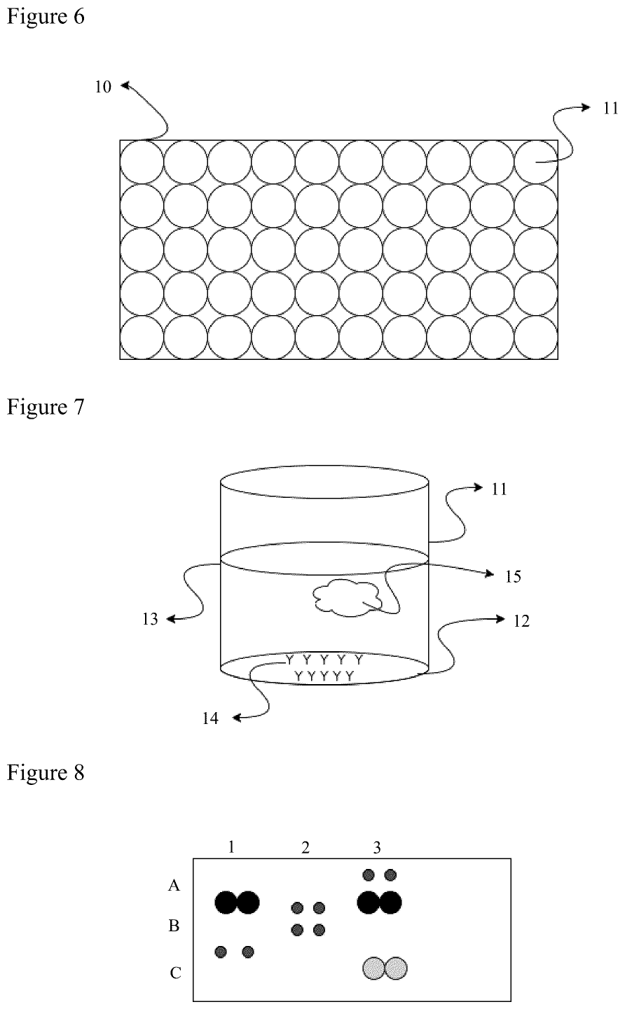

As shown in FIG. 6, at least one embodiment of the presently claimed invention may take the form of a cytokine assay kit 10 having separate wells 11. As further detailed in FIG. 7, the wells 11 comprise a microplate 12 coated in various antigens 14 and are filled with an enzyme substrate 13, all interacting with a plasma sample 15, the results of which can be gleaned from the panel array of FIG. 8.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

Moreover, in the description above, for the purposes of explanation, numerous specific details have been set forth in order to provide a thorough understanding of the embodiments. It will be apparent however, to one skilled in the art, that one or more other embodiments may be practiced without some of these specific details. The particular embodiments described are not provided to limit the invention but to illustrate it. The scope of the invention is not to be determined by the specific examples provided above but only by the claims below. It should also be appreciated that reference throughout this specification to “one embodiment”, “an embodiment”, “one or more embodiments”, or “different embodiments”, for example, means that a particular feature may be included in the practice of the invention. Similarly, it should be appreciated that in the description various features are sometimes grouped together in a single embodiment, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the detailed description are hereby expressly incorporated into this detailed description, with each claim standing on its own as a separate embodiment of the invention.

### REFERENCES

Johne H A, Frothingham L. Ein Eigenthuemlicher Fall von Tuberculose beim Rind. Deutsche Zeitschrift fuer Tiermedizin und Vergleichende Pathologie. 1895; 21:438-54.

Twort F W, Ingram G L, Ingram Y. A method for isolating and cultivating Mycobacterium enteridis chronicae pseudotuberculosae bovis johne and some experiments on the preparation of a diagnostic vaccine pseudotuberculosae enteritis of bovines. Proc Royal Soc Lond. 1912; 84:517-43.

Dalziel T K. Chronic Interstitial Enteritis. Br Med J. 1913; 2:1068-70.

Over K, Crandall P G, O'Bryan C A, et al. Current perspectives on Mycobacterium avium subsp. paratuberculosis, Johne's disease, and Crohn's disease: A review. Crit Rev Microbiol. 2011; 37:141-156.

Feller M, Huwiler K, Stephan R, Altpeter E, Shang A, Furrer H, Pfyffer G E, Jemmi T, Baumgartner A, Egger M. Mycobacterium avium subspecies paratuberculosis and Crohn's disease: a systematic review and meta-analysis. Lancet Infect Dis 2007; 7:607-613.

Abubakar I, Myhill D, Aliyu S H, Hunter P R. Detection of Mycobacterium avium subspecies paratuberculosis from patients with Crohn's disease using nucleic acid-based techniques: a systematic review and meta-analysis. Inflamm Bowel Dis 2008; 14:401-410.

Kuenstner J T, Naser S, Chamberlin W, Borody T J, Graham D Y, McNees A, Hermon-Taylor J, Hermon-Taylor A, Dow C T, Thayer W, Biesecker J, Collins M, Sechi L, Singh S V, Zhang P, Shafran I, et al. The Consensus on Mycobacterium avium ssp. paratuberculosis (MAP) 2017. Frontiers in Public Health September 2017; 5: article 208. doi: 10.3389/fpubh.2017.00208.

Graham D, Hardi R, Welton T, et al. Phase III randomized, double-blind, placebo-controlled, multicenter, parallel-group study to assess the efficacy and safety of add-on fixed-dose anti-mycobacterial therapy (RHB-104) in moderately to severely active Crohn's disease. United Eur Gastroenterol. 2018; 6:1586.

Kuenstner J T, Potula R, Bull T, et al. Presence of infection by Mycobacterium avium subsp. paratuberculosis in the blood of patients with Crohn's disease and control subjects shown by multiple laboratory culture and antibody methods. Microorganisms. 2020; 8:2054.

Peilin Zhang, U.S. Pat. No. 10,656,153, “Diagnostic Method and Devices for Autoimmune Disease,” granted Feb. 23, 2018.

Kuenstner J T, Xu Q, Bull T, Foddai A C G, Grant I R, Naser S, Potula R, Zhang P, Shafran I, Akhanli S A, Khaiboullina S, Kruzelock R. Cytokine expression in subjects with Mycobacterium avium ssp. paratuberculosis positive blood cultures and a meta-analysis of cytokine expression in Crohn's disease. Frontiers in Cellular and Infection Microbiology, 2024.

Kuenstner J T, Zhang Peilin, Potula Raghava, Galarneau Jean-Michel, Bach Horacio. Human antibodies against Mycobacterium avium ssp. paratuberculosis combined with cytokine levels for the diagnosis and selection of Crohn's disease patients for anti-mycobacterial therapy-A pilot study. PloS ONE 2024; 19 (10).

Graham David Y., Naser Saleh A., Borody Thomas, Hebzda Zbigniew, Sarles Harry, Levenson Scott, Hardi Robert, Arlukowicz Tomasz, Svorcan petar, Fathi Reza, Bibliowicz Aida, Anderson Patricia, McLean Patrick, Fehrmann Clara, Harris M. Scott, Zhao Shuhong, Kalfus ira N. Randomized, Double-Blind, Placebo-Controlled Study of Anti-mycobacterial Therapy (RHB-104) in Active Crohn's Disease. Antibiotics 2024, 13, 694.

What is claimed is:

1. A method of diagnosing a symptomatic or asymptomatic human with one or more mycobacterial diseases using a serological test, comprising:
Collecting a sample of said patient's blood;
Administering to said sample a combination of reagents that test for diagnostic cytokines and human antibodies;
Performing a diagnostic assay on said sample; and
Diagnosing said human with one or more mycobacterial diseases based on the results of said assay.

2. The method of claim 1, wherein said mycobacterial disease further comprises Mycobacterium avium subsp. paratuberculosis (MAP).

3. The method of claim 1, wherein said mycobacterial disease is one or more selected from a group consisting of: Crohn's disease (CD), tuberculosis (TB), and Johne's disease (JD).

4. The method of claim 3, wherein said tuberculosis (TB) is one or more selected from a group consisting of: tuberculosis (TB), latent tuberculosis (LTB), pulmonary tuberculosis (PTB), and Mycobacterium tuberculosis (MTB).

5. The method of claim 1, wherein said combination of human antibodies are one or more antibodies to MAP antigens.

6. The method of claim 5 wherein said human antibodies to MAP further comprise a combination of one or both of Hsp65 and PknG.

7. The method of claim 5, wherein said human antibodies to MAP further comprise a combination of one or more of Hsp65, PknG, PtpA, CL1, and MAP IDEXX.

8. The method of claim 1, wherein said combination of diagnostic cytokines further comprises one or more of IL-8, IL-17a, and IFN-g.

9. The method of claim 1, wherein said human antibodies further comprise a combination of one or more of RPOB, EF-G, ATP5a and EF-Tu.

10. The method of claim 1, wherein said diagnostic assay is a combination serological test used to detect said human antibodies to MAP, said cytokine biomarkers, and other tuberculosis like diseases.

11. The method of claim 1, where said serological test is used for the diagnosis and selection of tuberculosis patients for anti-tuberculosis therapy.

12. The method of claim 1, wherein said combination of human antibodies are antibodies to Mtb and further comprise one or more of Ag85B, Rv1860, and PStS1.

13. The method of claim 1, wherein said combination of cytokines further comprises one or more of IFNg, IL-2, IL-5, IL-10, IL-17, GM-CSF, and TNFa.

14. The method of claim 1, wherein said diagnostic assay is a combination serological test used to detect said human antibodies to Mtb, said cytokine biomarkers, and other tuberculosis like diseases.

15. The method of claim 1, wherein said human antibodies are one or more selected from a group consisting of: Hsp65, PknG, PtpA, CL1, MAP IDEXX, RPOB, EF-G, ATP5a, EF-Tu, Ag85B, Rv1860, and PStS1 and wherein said combination of cytokines are one or more selected from a group consisting of: IL-6, IL-8, IL-17a, IFN-g, IL-2, IL-5, IL-10, IL-17, GM-CSF, and TNFa.

16. A kit for use in diagnosing one or more Mycobacterium diseases, the kit comprising:
A microplate coated in separate wells with various antigens of a mycobacterial disease;
A plasma sample derived from a symptomatic or asymptomatic human that is deposited into the various wells of said microplate;
An enzyme substrate is added to the separate wells of said microplate and will be used for an optical density measurement;
A panel array to test for diagnostic cytokine levels in said human sample; and
Positive and negative control materials.

17. The kit of claim 16, wherein said antigens are one or more selected from a group consisting of:
Hsp65, PknG, PtpA, CL1, MAP IDEXX, RPOB, EF-G, ATP5a, EF-Tu, Ag85B, Rv1860, and PStS1 and wherein said diagnostic cytokines are one or more selected from a group consisting of: IL-6, IL-8, IL-17a, IFN-g, IL-2, IL-5, IL-10, IL-17, GM-CSF, and TNFa.

18. The kit of claim 16 wherein said microplate is coated in separate wells with various antigens of a mycobacterial disease, incubated with Mycobacterium phlei extract in order to bind non-specific antigens, and washed with an antibody antigen conjugate to remove unbound antigen.
